(19) United States
(12) Patent Application Publication
(10) Pub. No.: US 2012/0276130 A1
(43) Pub. Date: Nov. 1, 2012

(54) DIAGNOSTIC AND THERAPEUTIC METHODS FOR RHEUMATIC HEART DISEASE BASED UPON GROUP A STREPTOCOCCUS MARKERS

(76) Inventors: Immaculada Margarit Y Ros, Siena (IT); Reguzzi Valerio, Siena (IT); Sohail Ahmed, Siena (IT); Guido Grandi, Siena (IT); Erika Bartolini, Siena (IT)

(21) Appl. No.: 13/502,825

(22) PCT Filed: Oct. 20, 2010
(86) PCT No.: PCT/IB10/54753

§ 371 Date: Jul. 20, 2012

(30) Foreign Application Priority Data
Oct. 20, 2009 (GB) 0918392.2

(51) Int. Cl. C40B 40/10; G01N 33/566; A61P 37/04; G01N 21/64; A61P 9/00; A61P 31/04; A61K 39/09; C40B 30/04

(52) U.S. Cl. 424/190.1; 424/244.1; 436/501; 506/9; 506/18; 435/7.92

(57) ABSTRACT

This invention is in the field of identifying patients having rheumatic heart disease (RHD) associated with Streptococcus pyogenes (Group A Streptococcus; GAS) infection and identifying patients at risk of developing RHD associated with GAS infection. The invention also provides methods and compositions for preventing and treating RHD associated with GAS infection.

### TECHNICAL FIELD

This invention is in the field of identifying patients having rheumatic heart disease (RHD) associated with Streptococcus pyogenes (Group A Streptococcus; GAS) infection and identifying patients at risk of developing RHD associated with GAS infection. The invention also provides methods and compositions for preventing and treating RHD associated with GAS infection.

### BACKGROUND ART

The human pathogen Group A Streptococcus (Streptococcus pyogenes, GAS) is widely recognized as a major cause of common pharyngitis. Infections with this bacterium can additionally result in severe invasive diseases as well as in non-suppurative autoimmune sequelae. Acute rheumatic fever (ARF) is a multifocal autoimmune disease occurring in 0.1-3% of individuals following untreated GAS infection.

ARF is diagnosed by the updated Jones criteria which were first published in 1944. According to the updated Jones criteria, a diagnosis of ARF can be made when two major criteria (migratory polyarthritis; carditis; subcutaneous nodules; erythema marginatum; Sydenham's chorea), or one major criterion plus two minor criteria (fever; arthralgia; raised erythocyte sedimentation rate or C reactive protein; leukocytosis; ECG showing features of heart block) are present, along with evidence of GAS infection.

The major clinically significant sequela of ARF is rheumatic heart disease (RHD). RHD can lead to serious cardiac involvement, with myocarditis or valvulitis leading to death or valve replacement. Throughout the developing world, RHD remains the leading cause of acquired heart disease in individuals <50 years of age. In the developed world, ARF and RHD are less common due to the availability of antibiotics to treat GAS infections. However, a resurgence of ARF and RHD was reported in several areas of the United States in the mid 1980s and has persisted in the intermountain area surrounding Salt Lake City, Utah.

Currently, tests such as ECG and echocardiogram are used to confirm that a patient has developed RHD following diagnosis of ARF. To date, no assays are available for identifying individuals having or at risk of developing RHD as a result of GAS infection.

### DISCLOSURE OF THE INVENTION

The invention concerns methods of identifying individuals having or at risk of developing RHD resulting from GAS infection. The invention also concerns protein arrays that can be used in such methods. The invention also provides methods and compositions for preventing and treating RHD associated with GAS infection.

### Diagnostic Methods

The invention provides a method of diagnosing rheumatic heart disease (RHD) associated with GAS infection in a patient, or of identifying a patient at risk of developing RHD associated with GAS infection, said method comprising the steps of:
a) contacting a biological sample from a patient with at least one GAS antigen under conditions appropriate for binding of any antibodies present in the biological sample to the at least one GAS antigen, and
b) comparing the reactivity of antibodies in the biological sample from the patient to the at least one GAS antigen with the reactivity of antibodies in a control biological sample from a healthy individual to the at least one GAS antigen,
wherein a lower reactivity in the biological sample from the patient compared to the control biological sample from a healthy individual is indicative that the patient is suffering from rheumatic heart disease (RHD) associated with GAS infection or that the patient is at risk of developing RHD associated with GAS infection.

In one aspect, the invention provides a method of diagnosing rheumatic heart disease (RHD) associated with GAS infection in a patient, or of identifying a patient at risk of developing RHD associated with GAS infection, said method comprising the steps of:
a) contacting a biological sample from a patient with at least one GAS antigen selected from the group comprising the amino acid sequences of

SEQ ID NO: 1
(GAS5),
SEQ ID NO: 2
(GAS5F),
SEQ ID NO: 3
(GAS25),
SEQ ID NO: 4
(GAS40),
SEQ ID NO: 5
(GAS57),
SEQ ID NO: 6
(GAS97),
SEQ ID NO: 7
(GAS380),
and
SEQ ID NO: 8
(SpeA),

or functional equivalents thereof, under conditions appropriate for binding of any antibodies present in the biological sample to the at least one GAS antigen or to the functional equivalents thereof;
b) assessing the reactivity of any antibodies in the biological sample from the patient bound to the at least one GAS antigen or to the functional equivalents thereof, and
c) comparing the reactivity in step b) with the reactivity of antibodies in a control biological sample from a healthy individual bound to the at least one GAS antigen or to the functional equivalents thereof,
wherein a lower reactivity in the biological sample from the patient compared to the reactivity in the control biological sample from a healthy individual is indicative that the patient is suffering from rheumatic heart disease (RHD) associated with GAS infection or that the patient is at risk of developing RHD associated with GAS infection.

The term “rheumatic heart disease (RHD)” covers conditions affecting the heart following acute rheumatic fever including damage to the mitral valve and/or the aortic valve, myocarditis and pericarditis.

Analysis of serum samples from patients affected by RHD and from healthy individuals has led to the surprising finding that sera from patients affected by RHD display significantly lower reactivity with certain GAS antigens compared to the reactivity of sera from healthy patients. These findings provide the first evidence that reactivity with GAS antigens can be used to discriminate between sera derived from healthy individuals and sera derived from patients suffering from RHD. Specifically, it has been found that sera derived from RHD patients display a lower reactivity with the eight GAS antigens that are identified in Table 1 below:

TABLE 1
GAS antigens employed in the diagnostic methods of the invention

| | Internal GAS | | |
|---|---|---|---|
| SEQ ID NO | ref. | Spy number | gi number |
| 1 | GAS5 | spy0019 | gi-15674263 |
| 2 | GAS5F | spy0019 | gi-15674263 |
| | | (fragment from | |
| | | amino acids | |
| | | 224-398) | |
| 3 | GAS25 | spy0167 | gi-15674372 |
| 4 | GAS40 | spy0269 | gi-15674449 |
| 5 | GAS57 | spy0416 | gi-15674549 |
| 6 | GAS97 | spy1801 | gi-15675636 |
| 7 | GAS380 | spy1813 | gi-15675644 |
| 8 | SpeA | spyM3_1301 | gi-21910837 |

Detection of low reactivity against these eight GAS antigens in patient samples compared with reactivity in control samples from healthy individuals can thus be used to diagnose RHD associated with GAS infection or to identify patients with an increased risk of developing RHD associated with GAS infection. Conversely, detection of antibody reactivity against these eight GAS antigens in a patient sample that is similar to the reactivity present in a control sample from a healthy individual is indicative that the patient is not suffering from RHD and is at lower risk of developing RHD associated with GAS infection.

The methods of the invention may comprise contacting the biological sample from the patient with 1, 2, 3, 4, 5, 6, 7 or all 8 of the GAS antigens recited above, or with functional equivalents thereof.

Where the biological sample from the patient is contacted with 2 of the GAS antigens, the methods may comprise contacting the sample with: SEQ ID NOS:1 and 2; SEQ ID NOS:1 and 3; SEQ ID NOS:1 and 4; SEQ ID NOS:1 and 5; SEQ ID NOS:2 and 3; SEQ ID NOS:2 and 4; SEQ ID NOS:2 and 5; SEQ ID NOS:3 and 4; SEQ ID NOS:3 and 5; SEQ ID NOS:4 and 5, or functional equivalents thereof. The methods may also comprise contacting the sample with SEQ ID NOS:1 and 6; SEQ ID NOS:1 and 7; SEQ ID NOS:1 and 8; SEQ ID NOS:2 and 6; SEQ ID NOS:2 and 7; SEQ ID NOS:2 and 8; SEQ ID NOS:3 and 6; SEQ ID NOS:3 and 7; SEQ ID NOS:3 and 8; SEQ ID NOS:4 and 6; SEQ ID NOS:4 and 7; SEQ ID NOS:4 and 8; SEQ ID NOS:5 and 6; SEQ ID NOS:5 and 7; SEQ ID NOS:5 and 8; SEQ ID NOS:6 and 7; SEQ ID NOS:6 and 8, or SEQ ID NOS:7 and 8, or functional equivalents thereof.

Where the biological sample from the patient is contacted with 3 of the GAS antigens, the methods may comprise contacting the sample with any combination of 3 of the GAS antigens of SEQ ID NOS: 1, 2, 3, 4, 5, 6, 7, and 8. For example, the methods may comprise contacting the sample with SEQ ID NOS: 1, 2 and 3; SEQ ID NOS: 1, 3 and 4; SEQ ID NOS: 1, 4 and 5; SEQ ID NOS: 2, 3 and 4; SEQ ID NOS: 2, 4 and 5; SEQ ID NOS: 3, 4 and 5, or functional equivalents thereof.

Where the biological sample from the patient is contacted with 4 of the GAS antigens, the methods may comprise contacting the sample with any combination of 4 of the GAS antigens of SEQ ID NOS: 1, 2, 3, 4, 5, 6, 7, and 8. For example, the methods may comprise contacting the sample with: SEQ ID NOS: 1, 2, 3 and 4; SEQ ID NOS:2, 3, 4 and 5; SEQ ID NOS: 1, 3, 4 and 5, or functional equivalents thereof.

Where the biological sample from the patient is contacted with 5 of the GAS antigens, the methods may comprise contacting the sample with any combination of 5 of the GAS antigens of SEQ ID NOS: 1, 2, 3, 4, 5, 6, 7, and 8. For example, the methods may comprise contacting the sample with SEQ ID NOS:1, 2, 3, 4 and 5, or functional equivalents thereof.

Where the biological sample from the patient is contacted with 6 of the GAS antigens, the methods may comprise contacting the sample with any combination of 6 of the GAS antigens of SEQ ID NOS: 1, 2, 3, 4, 5, 6, 7, and 8.

Where the biological sample from the patient is contacted with 7 of the GAS antigens, the methods may comprise contacting the sample with: SEQ ID NOS: 1, 2, 3, 4, 5, 6, and 7; SEQ ID NOS: 1, 3, 4, 5, 6, 7 and 8; SEQ ID NOS: 1, 2, 4, 5, 6, 7 and 8; SEQ ID NOS: 1, 2, 3, 5, 6, 7 and 8; SEQ ID NOS: 1, 2, 3, 4, 6, 7 and 8; SEQ ID NOS: 1, 2, 3, 4, 5, 7 and 8; SEQ ID NOS: 1, 2, 3, 4, 5, 6 and 8; or functional equivalents thereof.

Alternatively, the biological sample from the patient may be contacted with all 8 of the GAS antigens, i.e. with SEQ ID NOS:1, 2, 3, 4, 5, 6, 7 and 8, or functional equivalents thereof.

The reactivity of antibodies bound to 1, 2, 3, 4, 5, 6, 7 or all 8 of these GAS antigens or functional equivalents thereof in the biological sample from a patient is compared to the reactivity of antibodies binding to these GAS antigens in a control biological sample from a healthy individual. The control biological sample from a healthy individual is contacted with the same combination of GAS antigens as the patient biological sample. Generally, average reactivities of antibodies bound to combinations of these GAS antigens will already have been determined in control biological samples from healthy individuals. Suitable methods for assessment the antibody reactivity are known in the art and are described in more detail below.

### Antibody Detection:

The methods of the invention described above all comprise the assessment of antibody reactivity, i.e. the detection of antibodies bound to the GAS antigens and of the titres of these antibodies. Methods for detecting antibodies bound to antigens and of determining antibody titres are well known to those of skill in the art and any such methods may be used.

For example, the GAS antigen or antigens (or functional equivalent) may be immobilised at known locations on a surface, such as on the surface of an array as described below. The immobilised antigens may be incubated with the immobilised antigens under conditions that allow the binding of any antibodies present in the sample to the antigens. A suitable incubation period may be around 1 hour. Following washing to remove any unbound antibodies, the detection of antibodies bound to the antigens may be accomplished using an entity that will bind and recognise the bound antibodies.

For example, the step of assessing the reactivity of any antibodies bound to the GAS antigens in any of the methods described above may comprise contacting the biological sample and GAS antigens with a labelled secondary antibody, such as a labelled anti-IgG antibody, under conditions suitable for the binding of the secondary antibody to any antibodies in the biological sample that have bound to the immobilised GAS antigens.

The secondary antibody, such as the anti-IgG antibody, may be labelled with a fluorescent or an enzyme label such that the binding of the secondary antibody, and thus the presence of antibodies against the GAS antigens in the biological sample, is detected by detecting the label. Where the label is a fluorescent label, comparison of fluorescence intensity may be used to assess relative antibody reactivity and thus determine whether there is a particular patient sample displays a lower antibody reactivity than a control biological sample. The background fluorescence intensity may be expected to be around 5,000. Taking into account standard deviation, a fluorescence intensity of at least 15,000 may be indicative of the presence of an antibody in the sample bound to the GAS antigen. A fluorescence intensity of at least 30,000 may be regarded as indicative of a high reactivity indicative of a high titre of antibodies bound to the GAS antigen in the sample. In some aspects of the invention, a fluorescence intensity of between 15,000 and 30,000 may thus be indicative of a low reactivity likely to be associated with RHD.

The methods described above may be conducted on a protein array, such as the arrays described in more detail below or using standard ELISA or dotblot techniques.

### Biological Samples:

The biological samples that may be tested in the methods of the invention may be any sample known to contain antibodies against GAS antigens. Examples of suitable samples are saliva samples, blood samples or serum samples. In particular, the sample may be a serum sample.

The biological sample from the patient is from a human patient. The human patient may be an adult, an adolescent between the ages of around 12 to around 18 or from a child under 12. The patient may be displaying clinical symptoms of acute rheumatic disease, including migratory polyarthritis; carditis; subcutaneous nodules; erythema marginatum; Sydenham's chorea, fever; arthralgia; raised erythocyte sedimentation rate or C reactive protein; leukocytosis; or ECG showing features of heart block. The patient may be displaying evidence of current GAS infection. In some cases, the patient may be asymptomatic for current GAS infection and acute rheumatic disease.

The control biological sample may be from a healthy individual from an equivalent geographical location as the biological sample from the patient.

The methods of the invention may be conducted in vitro. The methods of the invention may further comprise the step of obtaining the biological sample from the patient.

### Protein Arrays:

In order to facilitate the screening of biological samples against multiple GAS antigens simultaneously, the GAS antigens employed in the methods of the invention may be displayed on one or more protein arrays. For example, each GAS antigens may be displayed on a separate array or a single array may display multiple GAS antigens simultaneously. According to a further aspect of the invention, protein arrays are provided. These arrays are suitable for use in any of the methods described above.

The invention provides a protein array comprising at least two GAS antigens having an amino acid sequence selected from SEQ ID NO: 1, SEQ ID NO:2, SEQ ID NO:3, SEQ ID NO:4, SEQ ID NO:5, SEQ ID NO:6, SEQ ID NO:7 or SEQ ID NO:8, or a functional equivalent thereof.

The protein array may comprise 2, 3, 4, 5, 6, 7 or all 8 of these GAS antigens or functional equivalents thereof.

When the array comprises 2 of the GAS antigens, it may comprise antigens comprising the amino acid sequences of: SEQ ID NOS:1 and 2; SEQ ID NOS:1 and 3; SEQ ID NOS:1 and 4; SEQ ID NOS:1 and 5; SEQ ID NOS:2 and 3; SEQ ID NOS:2 and 4; SEQ ID NOS:2 and 5; SEQ ID NOS:3 and 4; SEQ ID NOS:3 and 5; SEQ ID NOS:4 and 5, or functional equivalents thereof. The array may alternatively comprise antigens comprising the amino acid sequences of SEQ ID NOS:1 and 6; SEQ ID NOS:1 and 7; SEQ ID NOS:1 and 8; SEQ ID NOS:2 and 6; SEQ ID NOS:2 and 7; SEQ ID NOS:2 and 8; SEQ ID NOS:3 and 6; SEQ ID NOS:3 and 7; SEQ ID NOS:3 and 8; SEQ ID NOS:4 and 6; SEQ ID NOS:4 and 7; SEQ ID NOS:4 and 8; SEQ ID NOS:5 and 6; SEQ ID NOS:5 and 7; SEQ ID NOS:5 and 8; SEQ ID NOS:6 and 7; SEQ ID NOS:6 and 8, or SEQ ID NOS:7 and 8, or functional equivalents thereof.

Where the array comprises 3 GAS antigens, it may comprise any combination of 3 of the GAS antigens of SEQ ID NOS: 1, 2, 3, 4, 5, 6, 7, and 8. For example, the array may comprise the GAS antigens of: SEQ ID NOS: 1, 2 and 3; SEQ ID NOS: 1, 3 and 4; SEQ ID NOS: 1, 4 and 5; SEQ ID NOS:, 2, 3 and 4; SEQ ID NOS: 2, 4 and 5; SEQ ID NOS: 3, 4 and 5, or functional equivalents thereof.

Where the array comprises 4 GAS antigens, it may comprise any combination of 4 of the GAS antigens of SEQ ID NOS: 1, 2, 3, 4, 5, 6, 7, and 8. For example, the array may comprise the GAS antigens of: SEQ ID NOS: 1, 2, 3 and 4; SEQ ID NOS:2, 3, 4 and 5; SEQ ID NOS: 1, 3, 4 and 5, or functional equivalents thereof.

Where the array comprises 5 GAS antigens, it may comprise any combination of 5 of the GAS antigens of SEQ ID NOS: 1, 2, 3, 4, 5, 6, 7, and 8. For example, the array may comprise the GAS antigens of: SEQ ID NOS:1, 2, 3, 4 and 5, or functional equivalents thereof.

Where the array comprises 6 GAS antigens, it may comprise any combination of 6 of the GAS antigens of SEQ ID NOS: 1, 2, 3, 4, 5, 6, 7, and 8.

Where the array comprises 7 GAS antigens, it may comprise the GAS antigens of: SEQ ID NOS: 1, 2, 3, 4, 5, 6, and 7; SEQ ID NOS: 1, 3, 4, 5, 6, 7 and 8; SEQ ID NOS: 1, 2, 4, 5, 6, 7 and 8; SEQ ID NOS: 1, 2, 3, 5, 6, 7 and 8; SEQ ID NOS: 1, 2, 3, 4, 6, 7 and 8; SEQ ID NOS: 1, 2, 3, 4, 5, 7 and 8; SEQ ID NOS: 1, 2, 3, 4, 5, 6 and 8; or functional equivalents thereof.

Alternatively, the array may comprise all 8 of the GAS antigens, i.e. SEQ ID NOS:1, 2, 3, 4, 5, 6, 7 and 8, or functional equivalents thereof.

The protein array may comprise additional GAS antigens.

Any type of protein array known in the art may be used in the method of invention. Production of protein arrays is described in Cretich, M., Damin F., et al (Biomolecular Engineering 23, 77-88 (2006)) and Zhu, H & Snyder, M. (Current Opinion in Chemical Biology, 7:55-63 (2003)).

For example, the protein array may be a glass slide to which the antigen or antigens are anchored. In its simplest form, the array may be a glass slide displaying a simple antigen prepared simply by coating glass microscope slides with aminosilane (Ansorge, Faulstich), adding an antigen-containing solution to the slide and drying. Slides coated with aminosilane may be obtained from Telechem and Pierce for coating with the antigen.

Alternatively, the array may display multiple antigens. For example, nitrocellulose-coated slides may be spotted with nanoliters of multiple GAS antigens. Such arrays may display replicates of each GAS antigen. The antigens spots in such arrays may be approximately 150 μm in diameter and contain ˜0.35 ng of protein

Other types of protein array include a 3D gel pad and microwell arrays. As will be apparent to the skilled reader, types of protein array that have not yet been conceived but which are devised in the future may well prove to be suitable for use in accordance with the present invention.

The invention further provides a kit comprising a protein array according to the invention and instructions for the use of the array in the diagnosis of patients having or at risk of developing rheumatic heart disease associated with GAS infection.

### Methods and Compositions for Treatment and Prevention of RHD

Currently, antibiotic prophylaxis (generally penicillin) is recommended for all patients diagnosed with ARF for a period of at least 5 years following diagnosis to reduce the risk of subsequent GAS infection and the development of RHD. The identification of which patients are at risk of RHD and which are not at risk of RHD allows tailoring of medical treatment for patients who have been diagnosed with ARF.

The invention that provides that, where a patient is identified by the method of the invention as suffering from RHD associated with GAS infection having an increased risk of developing RHD associated with GAS infection, the patient may be treated with antibiotics. Conversely, where a patient is identified by the method of the invention as having a low risk of developing RHD associated with GAS infection, antibiotic treatment may not be necessary.

The realization by the inventors that the sera from healthy individuals display high reactivity with the GAS antigens discussed above suggests that antibodies against these GAS antigens may play a protective role in preventing the development of RHD. The invention therefore provides a composition comprising at least one GAS antigen selected from the group comprising the amino acid sequences of SEQ ID NO:1, 2, 3, 4, 5, 6, 7 or 8, or a functional equivalent thereof. The invention also provides a composition comprising at least one antibody that binds specifically to at least one GAS antigen selected from the group comprising the amino acid sequences of SEQ ID NO:1, 2, 3, 4, 5, 6, 7 or 8, or a functional equivalent thereof. Theses compositions may be immunogenic compositions, e.g. vaccine compositions.

According to a further aspect, the invention provides a method of treating or preventing RHD associated with GAS infection comprising administering to a patient in need thereof at least one GAS antigen selected from the group comprising the amino acid sequences of SEQ ID NO:1, 2, 3, 4, 5, 6, 7 or 8, or a functional equivalent thereof. The invention further provides at least one GAS antigen selected from the group comprising the amino acid sequences of SEQ ID NO:1, 2, 3, 4, 5, 6, 7 or 8, or a functional equivalent thereof for use in treating or preventing RHD associated with GAS infection. The invention also provides the use of at least one GAS antigen selected from the group comprising the amino acid sequences of SEQ ID NO:1, 2, 3, 4, 5, 6, 7 or 8 or a functional equivalent thereof, in the manufacture of a medicament for treating or preventing RHD associated with GAS infection. Alternatively, nucleic acid molecules encoding these GAS antigens may be used.

The invention also provides a method of treating or preventing RHD associated with GAS infection comprising administering to a patient in need thereof at least one antibody that binds specifically to at least one GAS antigen selected from the group comprising the amino acid sequences of SEQ ID NO:1, 2, 3, 4, 5, 6, 7 or 8, or a functional equivalent thereof. The invention further provides at least one antibody that binds specifically to at least one GAS antigen selected from the group comprising the amino acid sequences of SEQ ID NO:1, 2, 3, 4, 5, 6, 7 or 8, or a functional equivalent thereof for use in treating or preventing RHD associated with GAS infection. The invention also provides the use of at least one antibody that binds specifically to at least one GAS antigen selected from the group comprising the amino acid sequences of SEQ ID NO:1, 2, 3, 4, 5, 6, 7 or 8 or a functional equivalent thereof, in the manufacture of a medicament for treating or preventing RHD associated with GAS infection.

Antibodies of the invention will typically bind specifically to the GAS antigen with an affinity of 1 μM, 100 nM, 10 nM, 1 nM, 100 μM or tighter. The term “antibody” includes intact immunoglobulin molecules, as well as fragments thereof which are capable of binding a polypeptide. These include hybrid (chimeric) antibody molecules [1, 2]; F(ab′)2 and F(ab) fragments and Fv molecules; non-covalent heterodimers [3, 4]; single-chain Fv molecules (sFv) [5]; dimeric and trimeric antibody fragment constructs; minibodies [6, 7]; humanized antibody molecules [8-10]; and any functional fragments obtained from such molecules, as well as antibodies obtained through non-conventional processes such as phage display. In some embodiments, the antibodies are monoclonal antibodies. Methods of obtaining monoclonal antibodies are well known in the art. In some embodiments the antibodies are humanised or fully-human antibodies.

The compositions and methods of treatment of the invention may employ 1, 2, 3, 4, 5, 6, 7, or all 8 of the GAS antigens discussed above, or antibodies that bind specifically to 1, 2, 3, 4, 5, 6, 7, or all 8 of these GAS antigens. Combinations of GAS antigens and antibodies binding specifically to these antigens may be used.

Examples of combinations of GAS antigens that may be used in the compositions and methods of treatment of these aspect of the invention include SEQ ID NOS:1 and 2; SEQ ID NOS:1 and 3; SEQ ID NOS:1 and 4; SEQ ID NOS:1 and 5; SEQ ID NOS:2 and 3; SEQ ID NOS:2 and 4; SEQ ID NOS:2 and 5; SEQ ID NOS:3 and 4; SEQ ID NOS:3 and 5; SEQ ID NOS:4 and 5; SEQ ID NOS:1 and 6; SEQ ID NOS:1 and 7; SEQ ID NOS:1 and 8; SEQ ID NOS:2 and 6; SEQ ID NOS:2 and 7; SEQ ID NOS:2 and 8; SEQ ID NOS:3 and 6; SEQ ID NOS:3 and 7; SEQ ID NOS:3 and 8; SEQ ID NOS:4 and 6; SEQ ID NOS:4 and 7; SEQ ID NOS:4 and 8; SEQ ID NOS:5 and 6; SEQ ID NOS:5 and 7; SEQ ID NOS:5 and 8; SEQ ID NOS:6 and 7; SEQ ID NOS:6 and 8, or SEQ ID NOS:7 and 8; SEQ ID NOS: 1, 2 and 3; SEQ ID NOS: 1, 3 and 4; SEQ ID NOS: 1, 4 and 5; SEQ ID NOS:, 2, 3 and 4; SEQ ID NOS: 2, 4 and 5; SEQ ID NOS: 3, 4 and 5; SEQ ID NOS: 1, 2, 3 and 4; SEQ ID NOS:2, 3, 4 and 5; SEQ ID NOS: 1, 3, 4 and 5; SEQ ID NOS:1, 2, 3, 4 and 5; SEQ ID NOS: 1, 2, 3, 4, 5, 6, and 7; SEQ ID NOS: 1, 3, 4, 5, 6, 7 and 8; SEQ ID NOS: 1, 2, 4, 5, 6, 7 and 8; SEQ ID NOS: 1, 2, 3, 5, 6, 7 and 8; SEQ ID NOS: 1, 2, 3, 4, 6, 7 and 8; SEQ ID NOS: 1, 2, 3, 4, 5, 7 and 8; SEQ ID NOS: 1, 2, 3, 4, 5, 6 and 8; or functional equivalents thereof. Antibodies binding to these combinations of GAS antigens may also be used.

The compositions and methods described above may be useful in the treatment and prevention of GAS infection in general, as well as in the treatment and prevention of RHD associated with GAS infection.

### Formulation of Compositions for Treatment and Prevention of RHD

As detailed above, compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s).

Compositions will generally be administered to a human in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more typical. Preservative-free vaccines are particularly favoured.

To improve thermal stability, a composition may include a temperature protective agent. Further details of such agents are provided below.

To control tonicity, it is typical to include a physiological salt, such as a sodium salt. Sodium chloride (NaCl) is generally used, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, more often between 240-360 mOsm/kg, and will more typically fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is typically sterile. The composition is also typically non-pyrogenic e.g. containing <1 EU (endotoxin unit, a standard measure) per dose, for example <0.1 EU per dose. The composition is often gluten free.

The composition may include material for a single immunisation, or may include material for multiple immunisations (i.e. a ‘multidose’ kit). The inclusion of a preservative is typical in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Compositions of the invention may also comprise one or more immunoregulatory agents. Often, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts (or mixtures thereof). Calcium salts include calcium phosphate (e.g. the “CAP” particles disclosed in ref. 11). Aluminum salts include hydroxides, phosphates, sulfates, etc., with the salts taking any suitable form (e.g. gel, crystalline, amorphous, etc.). Adsorption to these salts is often employed. The mineral containing compositions may also be formulated as a particle of metal salt [12].

The adjuvants known as aluminum hydroxide and aluminum phosphate may be used. These names are conventional, but are used for convenience only, as neither is a precise description of the actual chemical compound which is present (e.g. see chapter 9 of reference 13)). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, such as between 0.8 and 1.2, typically 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, such as between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are frequently sterile and pyrogen-free. A suspension may include free aqueous phosphate ions e.g. present at a concentration between 1.0 and 20 mM, such as between 5 and 15 mM, e.g. about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a mammal is typically less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

Aluminium phosphates are particularly preferred, particularly in compositions which include a H. influenzae saccharide antigen, and a typical adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where there is more than one conjugate in a composition, not all conjugates need to be adsorbed.

### B. Oil Emulsions

Oil emulsion compositions suitable for use as adjuvants in the invention include squalene-water emulsions, such as MF59 [Chapter 10 of ref. 13; see also ref. 14] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolizable) and biocompatible. The oil droplets in the emulsion are generally less than 5 μM in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, such as at least 15, e.g. at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, such as 0.1 to 10% and in particular 0.1 to 1% or about 0.5%.

Preferred emulsion adjuvants have an average droplets size of <1 μm e.g. ≦750 nm, ≦500 nm, ≦400 nm, ≦300 nm, ≦250 nm, ≦220 nm, ≦200 nm, or smaller. These droplet sizes can conveniently be achieved by techniques such as microfluidisation.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [15-17], as described in more detail in Chapter 10 of ref 18 and chapter 12 of ref 19. The MF59 emulsion advantageously includes citrate ions e.g. 10 mM sodium citrate buffer.
An emulsion of squalene, a tocopherol, and polysorbate 80 (Tween 80). The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is typically ≦1 as this provides a more stable emulsion. Squalene and Tween 80 may be present volume ratio of about 5:2 or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, often about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [20].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 m/ml Triton X-100 and 100 m/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [21] (0.05-1% Thr-MDP, 5% squalane, 2.5% Pluronic L121 and 0.2% polysorbate 80). It can also be used without the Thr-MDP, as in the “AF” adjuvant [22] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is generally thermoreversible and/or has at least 90% of the oil droplets (by volume) with a size less than 200 nm [23]. The emulsion may also include one or more of: alditol; a cryoprotective agent (e.g. a sugar, such as dodecylmaltoside and/or sucrose); and/or an alkylpolyglycoside. The emulsion may include a TLR4 agonist [24]. Such emulsions may be lyophilized.
An emulsion of squalene, poloxamer 105 and Abil-Care [25]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 26, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 27, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles [28].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [29].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [29].

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly humans (e.g. aged 60 years or older) because vitamin E has been reported to have a positive effect on the immune response in this patient group [30]. They also have antioxidant properties that may help to stabilize the emulsions [31]. A preferred α-tocopherol is DL-α-tocopherol, and the preferred salt of this tocopherol is the succinate. The succinate salt has been found to cooperate with TNF-related ligands in vivo.

### C. Saponin Formulations [Chapter 22 of Ref. 13]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. In some cases the saponin is QS21. A method of production of QS21 is disclosed in ref. 32. Saponin formulations may also comprise a sterol, such as cholesterol [33].

Combinations of saponins and cholesterols can be used to form unique particles called immunostimulating complexs (ISCOMs) [chapter 23 of ref. 13]. ISCOMs typically also include a phospholipid such as phosphatidylethanolamine or phosphatidylcholine. Any known saponin can be used in ISCOMs. In some embodiments, the ISCOM includes one or more of QuilA, QHA & QHC. ISCOMs are further described in refs. 33-35. Optionally, the ISCOMS may be devoid of additional detergent [36].

A review of the development of saponin based adjuvants can be found in refs. 37 & 38.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 39-44. Virosomes are discussed further in, for example, ref 45

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref 46. Such “small particles” of 3dMPL are small enough to be sterile filtered through a 0.22 μm membrane [46]. Other non-toxic LPS derivatives include monophosphoryl lipid A mimics, such as aminoalkyl glucosaminide phosphate derivatives e.g. RC-529 [47,48].

Lipid A derivatives include derivatives of lipid A from Escherichia coli such as OM-174. OM-174 is described for example in refs. 49 & 50.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 51, 52 and 53 disclose possible analog substitutions e.g. replacement of guanosine with 2′-deoxy-7-deazaguanosine. The adjuvant effect of CpG oligonucleotides is further discussed in refs. 54-59.

The CpG sequence may be directed to TLR9, such as the motif GTCGTT or TTCGTT [60]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 61-63. In some embodiments, the CpG is a CpG-A ODN.

In other embodiments, the CpG oligonucleotide is constructed so that the 5′ end is accessible for receptor recognition. Optionally, two CpG oligonucleotide sequences may be attached at their 3′ ends to form “immunomers”. See, for example, refs. 60 & 64-66.

A useful CpG adjuvant is CpG7909, also known as ProMune™ (Coley Pharmaceutical Group, Inc.). Another is CpG1826. As an alternative, or in addition, to using CpG sequences, TpG sequences can be used [67], and these oligonucleotides may be free from unmethylated CpG motifs. The immunostimulatory oligonucleotide may be pyrimidine-rich. For example, it may comprise more than one consecutive thymidine nucleotide (e.g. TTTT, as disclosed in ref. 67), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref. 67), and/or it may have a nucleotide composition with >25% cytosine (e.g. >35%, >40%, >50%, >60%, >80%, etc.). These oligonucleotides may be free from unmethylated CpG motifs. Immunostimulatory oligonucleotides will typically comprise at least 20 nucleotides. They may comprise fewer than 100 nucleotides.

A particularly useful adjuvant based around immunostimulatory oligonucleotides is known as IC-31™ [68]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 427). The polycationic polymer may be a peptide comprising 11-mer amino acid sequence KLKLLLLLKLK (SEQ ID NO: ⁴²⁶). The oligonucleotide and polymer can form complexes e.g. as disclosed in references 69 & 70.

Bacterial ADP-ribosylating toxins and detoxified derivatives thereof may be used as adjuvants in the invention. In some embodiments, the protein is derived from E. coli (E. coli heat labile enterotoxin “LT”), cholera (“CT”), or pertussis (“PT”). The use of detoxified ADP-ribosylating toxins as mucosal adjuvants is described in ref. 71 and as parenteral adjuvants in ref. 72. The toxin or toxoid is typically in the form of a holotoxin, comprising both A and B subunits. In some embodiments, the A subunit contains a detoxifying mutation; often the B subunit is not mutated. In some embodiments, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 73-80. A useful CT mutant is or CT-E29H [81]. Numerical reference for amino acid substitutions is typically based on the alignments of the A and B subunits of ADP-ribosylating toxins set forth in ref 82, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [83], etc.) [84], interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres [85] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [86].

### H. Microparticles

Microparticles may also be used as adjuvants in the invention. Microparticles (i.e. a particle of ˜100 nm to ˜150 μm in diameter, in some embodiments ˜200 nm to ˜30 μm in diameter, e.g. ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of Ref. 13)

Examples of liposome formulations suitable for use as adjuvants are described in refs. 87-89.

### J. Polyoxyethylene Ether and Polyoxyethylene Ester Formulations

Adjuvants suitable for use in the invention include polyoxyethylene ethers and polyoxyethylene esters [90]. Such formulations further include polyoxyethylene sorbitan ester surfactants in combination with an octoxynol [91] as well as polyoxyethylene alkyl ethers or ester surfactants in combination with at least one additional non-ionic surfactant such as an octoxynol [92]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Phosphazenes

A phosphazene, such as poly[di(carboxylatophenoxy)phosphazene] (“PCPP”) as described, for example, in references 93 and 94, may be used.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquimod (“R-837”) [95,96], Resiquimod (“R-848”) [97], and their analogs; and salts thereof (e.g. the hydrochloride salts). Further details about immunostimulatory imidazoquinolines can be found in references 98 to 102.

### N. Substituted Ureas

Substituted ureas useful as adjuvants include compounds of formula I, II or III, or salts thereof:

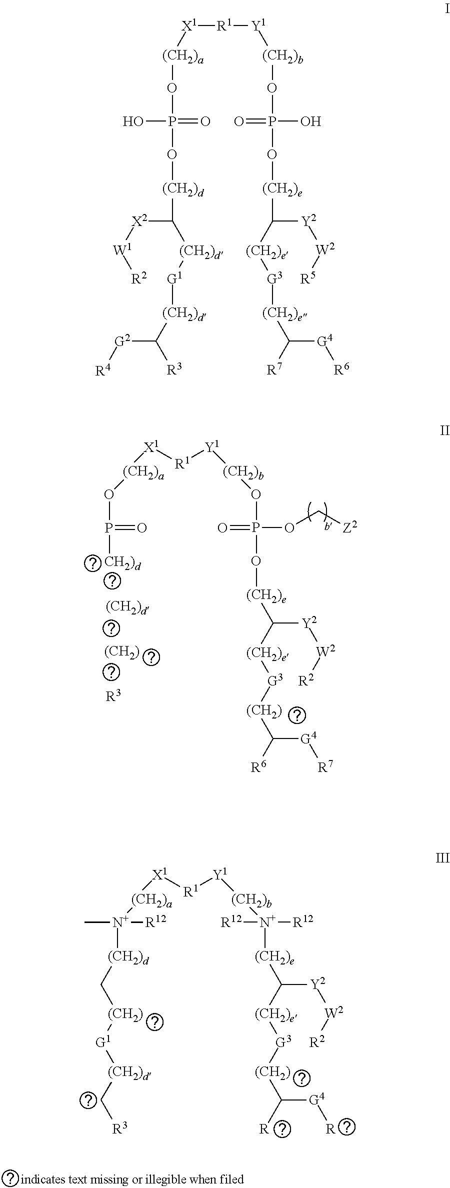

as defined in reference 103, such as ‘ER 803058’, ‘ER 803732’, ‘ER 804053’, ER 804058′, ‘ER 804059’, ‘ER 804442’, ‘ER 804680’, ‘ER 804764’, ER 803022 or ‘ER 804057’ e.g.:

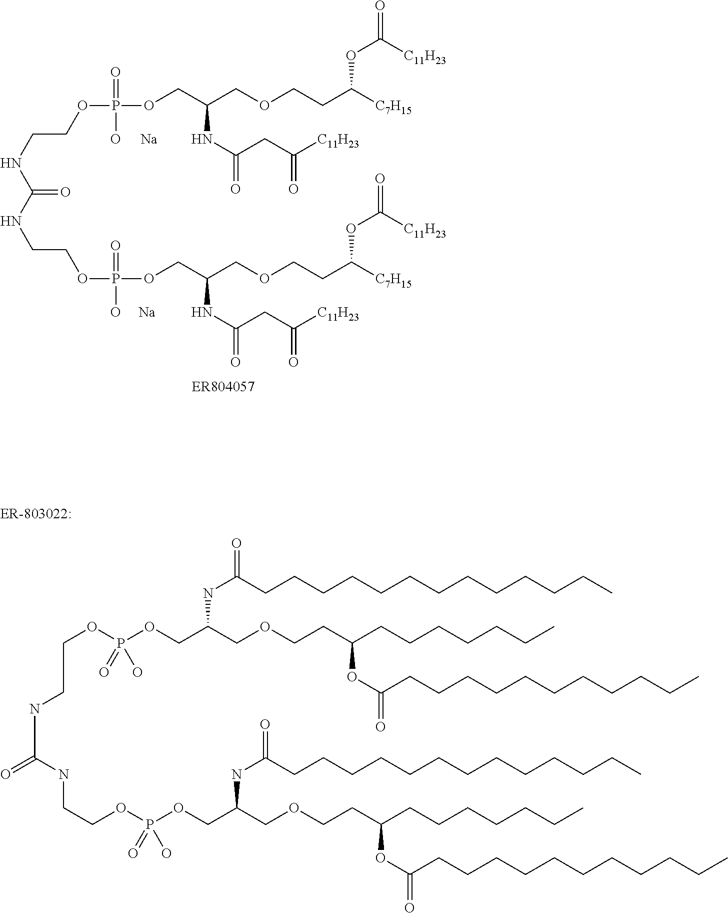

### O. Further Adjuvants

Further adjuvants that may be used with the invention include:
An aminoalkyl glucosaminide phosphate derivative, such as RC-529 [104,105].
A thiosemicarbazone compound, such as those disclosed in reference 106. Methods of formulating, manufacturing, and screening for active compounds are also described in reference 106. The thiosemicarbazones are particularly effective in the stimulation of human peripheral blood mononuclear cells for the production of cytokines, such as TNF-α.
A tryptanthrin compound, such as those disclosed in reference 107. Methods of formulating, manufacturing, and screening for active compounds are also described in reference 107. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

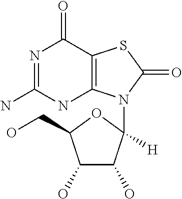

and prodrugs thereof; (b) ANA975; (c) ANA-025-1; (d) ANA380; (e) the compounds disclosed in references 108 to 110 Loxoribine (7-allyl-8-oxoguanosine) [111].
Compounds disclosed in reference 112, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [113,114], Hydrapthalamide compounds, Benzophenone compounds, Isoxazole compounds, Sterol compounds, Quinazilinone compounds, Pyrrole compounds [115], Anthraquinone compounds, Quinoxaline compounds, Triazine compounds, Pyrazalopyrimidine compounds, and Benzazole compounds [116].
Compounds containing lipids linked to a phosphate-containing acyclic backbone, such as the TLR4 antagonist E5564 [117,118]:
A polyoxidonium polymer [119,120] or other N-oxidized polyethylene-piperazine derivative.
Methyl inosine 5′-monophosphate (“MIMP”) [121].
A polyhydroxlated pyrrolizidine compound [122], such as one having formula:

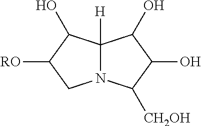

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A CD1d ligand, such as an α-glycosylceramide [123-130] (e.g. α-galactosylceramide), phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″−O-sulfo-galactosylceramide, etc.
A gamma inulin [131] or derivative thereof, such as algammulin.

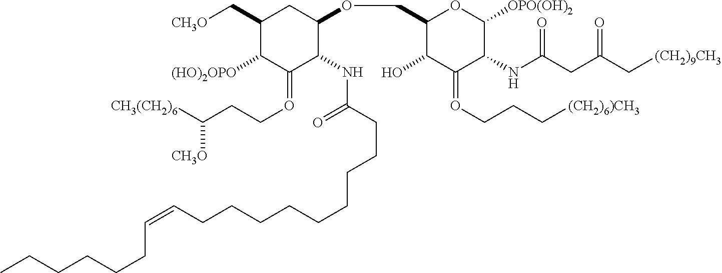

### Adjuvant Combinations

The invention may also comprise combinations of one or more of the adjuvants identified above. For example, the following adjuvant compositions may be used in the invention: (1) a saponin and an oil-in-water emulsion [132]; (2) a saponin (e.g. QS21)+a non-toxic LPS derivative (e.g. 3dMPL) [133]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [134]; (5) combinations of 3dMPL with, for example, QS21 and/or oil-in-water emulsions [135]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 13.

The use of an aluminium hydroxide and/or aluminium phosphate adjuvant is typical, and antigens are generally adsorbed to these salts. Calcium phosphate is another typical adjuvant. Other adjuvant combinations include combinations of Th1 and Th2 adjuvants such as CpG & alum or resiquimod & alum. A combination of aluminium phosphate and 3dMPL may be used.

The compositions of the invention may elicit both a cell mediated immune response as well as a humoral immune response. This immune response may induce long lasting (e.g. neutralising) antibodies and a cell mediated immunity that can quickly respond upon exposure to pnuemococcus.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. In some embodiments, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are typical TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. In some embodiments, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are typical TH2 adjuvants for use in the invention.

In some embodiments, the invention includes a composition comprising a combination of a TH1 adjuvant and a TH2 adjuvant. Often, such a composition elicits an enhanced TH1 and an enhanced TH2 response, i.e., an increase in the production of both IgG1 and IgG2a production relative to immunization without an adjuvant. Generally, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. The immune response may provide for one or both of an enhanced TH1 response and an enhanced TH2 response.

The enhanced immune response may be one or both of a systemic and a mucosal immune response. The immune response may provide for one or both of an enhanced systemic and an enhanced mucosal immune response. Typically the mucosal immune response is a TH2 immune response. Typically the mucosal immune response includes an increase in the production of IgA.

The compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a mammal. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

As mentioned above, a composition may include a temperature protective agent, and this component may be particularly useful in adjuvanted compositions (particularly those containing a mineral adjuvant, such as an aluminium salt). As described in reference 136, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In one embodiment, the polyethylene glycol can have an average molecular weight of about 300 Da (PEG-300′).

The invention provides a composition comprising: (i) one or more antigen(s); and (ii) a temperature protective agent. This composition may be formed by mixing (i) an aqueous composition comprising one or more antigen(s), with (ii) a temperature protective agent. The mixture may then be stored e.g. below 0° C., from 0-20° C., from 20-35° C., from 35-55° C., or higher. It may be stored in liquid or frozen form. The mixture may be lyophilised. The composition may alternatively be formed by mixing (i) a dried composition comprising one or more antigen(s), with (ii) a liquid composition comprising the temperature protective agent. Thus component (ii) can be used to reconstitute component (i).

### Functional Equivalents:

The SEQ ID NOS used to identify the GAS antigens that may be used in the methods, protein arrays and medical uses of the invention described above are full length sequences for these GAS antigens.

The methods, protein arrays and medical uses of the invention are not limited to the use of these full-length GAS antigens but also encompass any “functional equivalent” of any of these GAS antigens.

The term “functional equivalent” as used herein is intended to encompass variants of the GAS antigens having the full-length sequences shown in the sequence listing that retain the ability to interact with antibodies against the full-length GAS antigen present in the biological and that may thus be used in place of the full-length GAS antigens.

The term “functional equivalent” thus encompasses fragments of the full-length GAS antigens having the sequences shown in the sequence listing. Such fragments may retain the ability to bind to antibodies that bind to the full-length GAS antigens. The functional equivalents of the invention may bind to antibodies generated against the full-length GAS antigen with an affinity of at least 10⁻⁷M.

Fragments include at least n consecutive amino acids of the full-length GAS antigen sequences, wherein n is 7 or more (e.g. 8, 10, 12, 14, 16, 18, 20, 25, 30, 35, 40, 50, 60, 70, 80, 90, 100, 150, 200, 250 or more). Fragments may comprise an epitope from the full-length GAS antigen sequence. Further fragments may lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the full-length sequence. For example, fragments that may be employed in the methods and arrays of the invention include fragments that are lacking the leader sequences and/or the transmembrane sequences present in the full-length GAS antigens.

Further examples of fragments that may be used in the methods and arrays of the invention include N-terminal fragments. Examples of such fragments include the amino acid sequence shown in SEQ ID NO:9 (which is an N-terminal fragment of the sequence in SEQ ID NO:5) and the amino acid sequence shown in SEQ ID NO:10 (which is an N-terminal fragment of SEQ ID NO:4).

The term “functional equivalent” also includes variants of the full-length GAS proteins having amino acid substitutions and fragments of such variants. Variants may have 50% or more identity (e.g. 60%, 65%, 70%, 75%, 80%, 85%, 90%, 91%, 92%, 93%, 94%, 95%, 96%, 97%, 98%, 99%, 99.5% or more) to the full-length GAS antigen sequences provided herein. Variants may contain conservative amino acid substitutions compared to the GAS antigen sequence given in the sequence listing. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

The term “functional equivalent” additionally encompasses longer variants of the GAS antigens including fusion proteins that include an additionally entity that has been chemically or genetically linked to the GAS antigen. For example, the GAS antigen may be attached a label that facilitates its localisation on a protein array or facilitates detecting it when it is bound to an antibody. Examples of such labels include an analytically-detectable reagent such as a radioisotope, a fluorescent molecule or an enzyme. Alternatively, the GAS antigen may be fused to a domain that facilitated its initial purification, such as a histidine or GST domain.

The term “functional equivalent” also includes mimetics of the GAS antigens, variants and fragments described above, which are structurally similar to the GAS antigens and retain the ability to bind to antibodies against the full-length GAS antigens.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### BRIEF DESCRIPTION OF THE DRAWINGS

FIG. 1. Age distribution of patients with rheumatic heart disease (RHD) and Yemeni healthy blood donors (YHD) from whom sera was collected. Age-matched sera samples selected for the study shown.

FIG. 2. Protein micro array set-up and validation. A, SDS-PAGE analysis of purified recombinant GAS proteins stained with Coomassie. Molecular weight markers in lane 1. B, Representative image of a chip after incubation with a human serum and with Cy3-labelled anti-human IgG and Cy5-labelled anti-human IgM. Replicates of tested antigens and of negative and positive IgG and IgM controls are highlighted. C, graphic representation of the control human IgG curve. The chip image of different IgG concentration revealed by incubation with anti-human IgG-Cy3 is shown below the graph. D, Sigmoid-derived data normalization method. Data were normalized using the sigmoid control curve (black) adjusted to a reference sigmoid curve (red; ₁d, ideal sigmoid curve; P and P′, intersection points of not normalized, Val, and normalized, N(Val), MFI values on the experimental and reference sigmoid curves; HL, values correspond to normalized MFI values of 30,000; LL are normalized MFI values of 15,000.

FIG. 3. Percentage of Yeminite and Italian healthy donor sera with high responses (MFI>30000) to GAS antigens. Antigens are represented in decreasing order responses.

FIG. 4. Comparison of the immuno reactivity of the 40 YHD (dark grey in FIG. 4) and 43 RHD (light grey in FIG. 4) age matched selected human sera. Normalized FI (MFI) values were subjected to unsupervised bi-dimensional hierarchical clustering using dedicated software (TIGR Multiexperiment Viewer (MeV) software (http://www.tigr.org/software/tm4/mev.html) to define the antigen recognition patterns of the two groups of sera, resulting in the identification of two major groups of highly recognized antigens (1 and 2 in FIG. 4A, also shown in FIG. 4B). This clustering analysis distributed the sera from higher reactivity (on the left) to lower reactivity (on the right). Two main groups of sera could be distinguished, a highly reactive group which comprised mainly sera from healthy donors (A in left of FIG. 4) and second group displaying lower reactivity and including mainly sera from RHD patients (group B in FIG. 4).

FIG. 5. Application of K-mean cluster analysis to classify the GAS antigens present in the chip in 10 clusters (KA1 to KA10) eliciting similar recognition patterns. Four of the antigen clusters (numbers KA1, KA5, KA9, KA10) identified contained antigens with higher fluorescence values than the remaining clusters. In cluster KA 1, the most reactive sera comprised a large number of YHD, suggesting the presence on the chip of a group of antigens, the reactivity of which allows discrimination between sera derived from healthy donors and sera from RHD patients.

FIG. 6. Identification of antigen clusters that enable discrimination between healthy and RHD patients. Sera from the KA1 cluster in FIG. 6 were further classified on the basis of their recognition profiles to different groups of antigens using a mono-dimensional hierarchical clustering analysis, allowing the definition of two sera clusters HS1 (violet box) and HS2 (blue box). The numbers of healthy sera and patient sera present or absent in each of the two clusters is reported in FIG. 6B. Most of the YHD can be found in the high reactivity, blue, HS2 cluster, while the majority of RHD sera are found in the low reactivity, violet HS1 cluster. The ability to distinguish between the two sera populations using this type of test was defined in terms of specificity and sensitivity (FIG. 6C). For this particular group of antigens, specificity and sensitivity values of 0.73 and 0.69 were obtained. FIG. 6C also shows the ideal theoretical example of maximum specificity and sensitivity (values of 1).

FIG. 7. The analysis described in FIG. 6 was applied to other antigen clusters: KA5 (B), KA9 (C), KA10 (D), KA5+M9 (E), GAS5+GAS5F+GAS25+GAS40 (F), GAS5+GAS5F+GAS25+GAS40+GAS57 (G), GAS5+GAS25+GAS40+GAS57 (H), GAS5F+GAS25+GAS40+GAS57 (I). The specificity and sensitivity values are shown for each cluster.

### MODES FOR CARRYING OUT THE INVENTION

### Introduction

We have developed a protein microarray containing 130 recombinant GAS protein antigens. The chip was instrumental for the selection of antigens eliciting high antibody responses in pharyngitis patients and also allowed to unveil high responses against GAS antigens in sera from patients with Tic disease, strongly supporting that GAS antigen-dependent induction of autoantibodies in susceptible individuals may be involved in the occurrence of tic disorders (Bombaci M, et al. 2009 PLoS ONE 4, 7: e6332. doi:10.1371).

Here we used the protein microarray to analyze the immune response against 130 recombinant GAS proteins in RHD patients and healthy donors, with the aim of identifying antigen recognition patterns allowing us to discriminate between the two populations. This approach led to the identification of a cluster of antigens highly recognized by healthy donors, but not by RHD patients, which can set the basis for a diagnosis test.

### Materials and Methods

### Human Sera

Rheumatic heart disease patient sera were collected from 60 male or female 11-40 years old patients from a Middle-East country (Yemen) presenting clinical symptoms of RHD.

Anti-GAS titers are known vary according to a number of factors, including age and geographic origin. In fact, anti GAS antibody titers in healthy people are low in early childhood, rise to a peak in children aged 5 to 15 years, decrease in late adolescence and early adulthood, and then flatten off after that. For this reason, comparison between rheumatic heart disease (RHD) sera and control Yemeni healthy donor (YHD) groups was performed using groups of the same age range (17-40 year old), thus excluding 11 to 16 year old RHD patient sera, for which control sera were not available. The final number of sera used in the comparison was 40 YHD and 43 RHD.

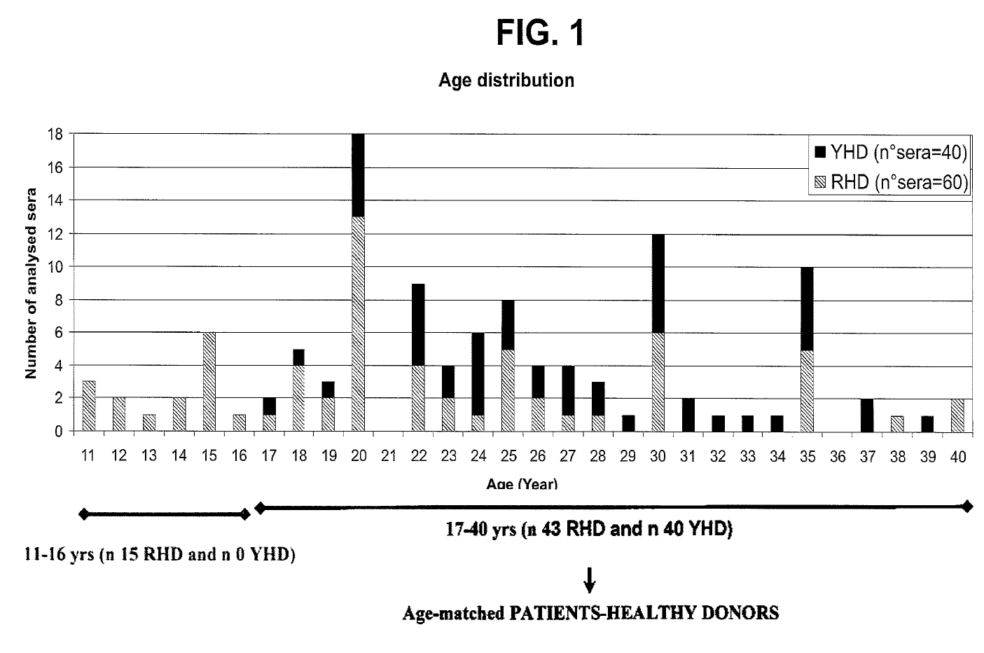

FIG. 1 shows the distribution analysis of the two populations available and those selected for the study.

In addition, a collection of 20 sera from healthy Italian human donors (IHD) was used for an additional comparison to healthy Yemenites, taking into consideration the higher use of antibiotic prophylaxis of GAS infections in the former western population.

All serum samples were residuals obtained during routine medical controls for RHD diagnosis or bloodlettings and were made available by the Department of Child and Adolescent Neuropsychiatry, University La Sapienza, Rome.

### GAS Protein Microarray

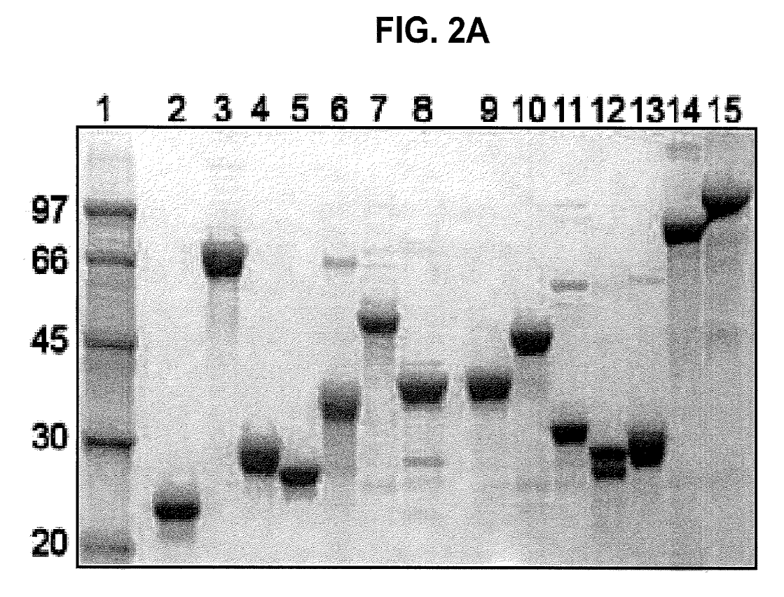

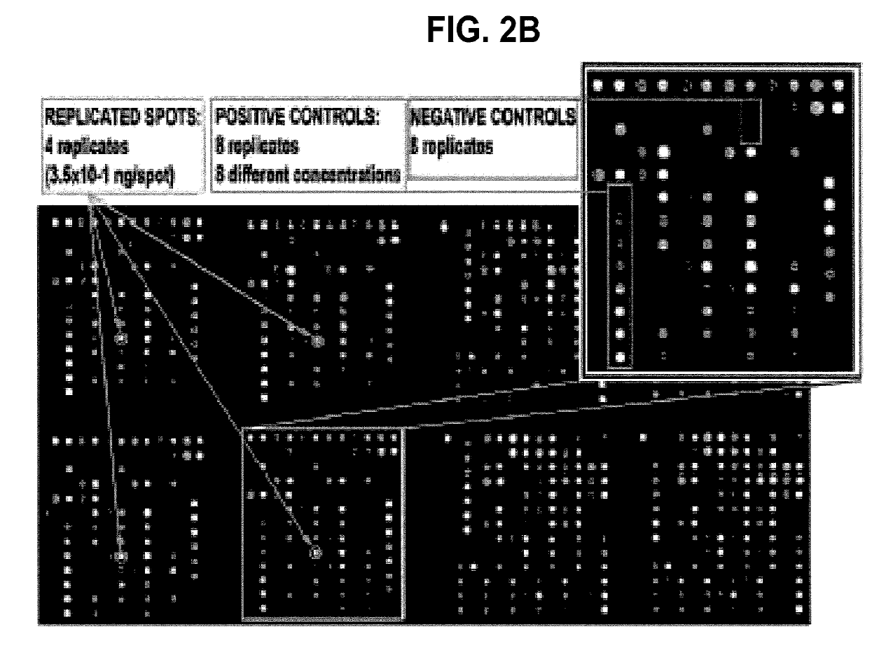

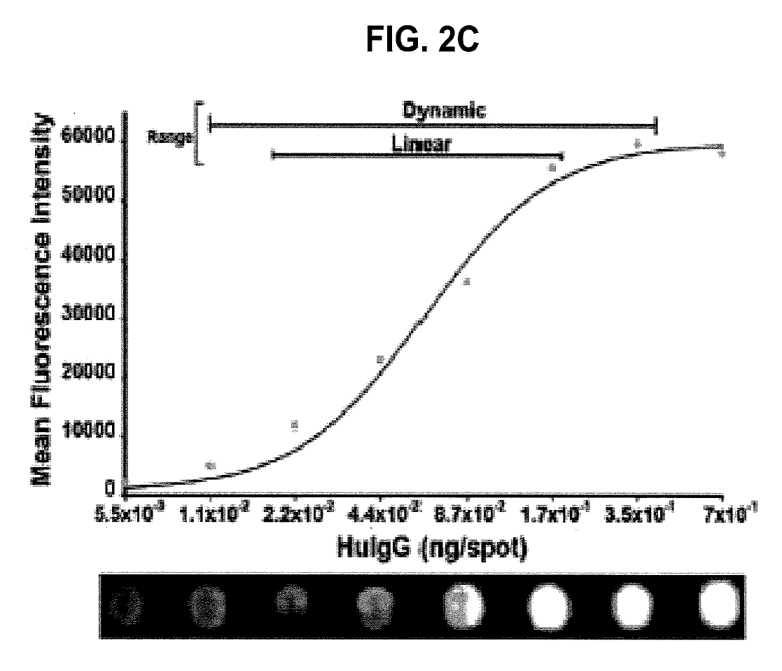

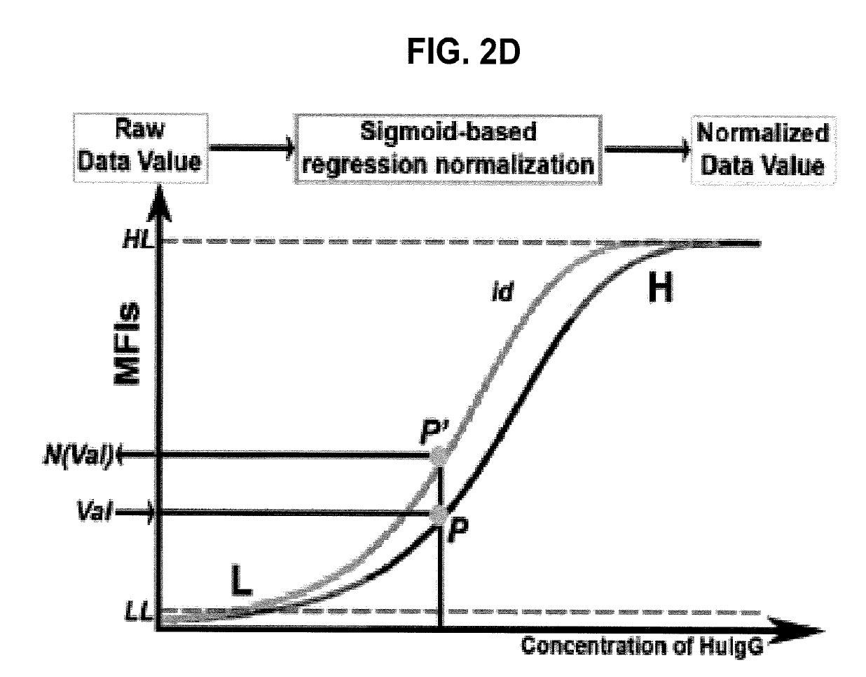

A protein array was generated by depositing on a nitrocellulose chip 130 recombinant proteins mainly selected from the GAS SF370 M1 genome (see FIG. 2 for details on chip set up).

The chips were incubated with the different sera and reactivity was evaluated by detecting total IgG bound to each deposited protein whit fluorescently labeled anti-human IgG and measuring the resulting Fluorescence Intensity (FI) values. For each slide, protein MFI values were normalized to a sigmoid adjusted standard IgG curve used as reference (FIG. 2).

Antigen recognition by tested sera was considered positive when MFI values were equal to or higher than 15,000, corresponding to the background value plus 2 standard deviations. MFI values equal to or above 30,000 were considered as high responses. The array was probed with 120 sera from patients (20 sera of Italian healthy donors (IHD), 40 of Yemenite healthy donors (YHD) and 60 of RHD Yemenite patients (RHD)).

### Results

GAS Antigens Recognized by Sera from Healthy Donors: Antibody Responses are Higher in Yemenites than in Italians

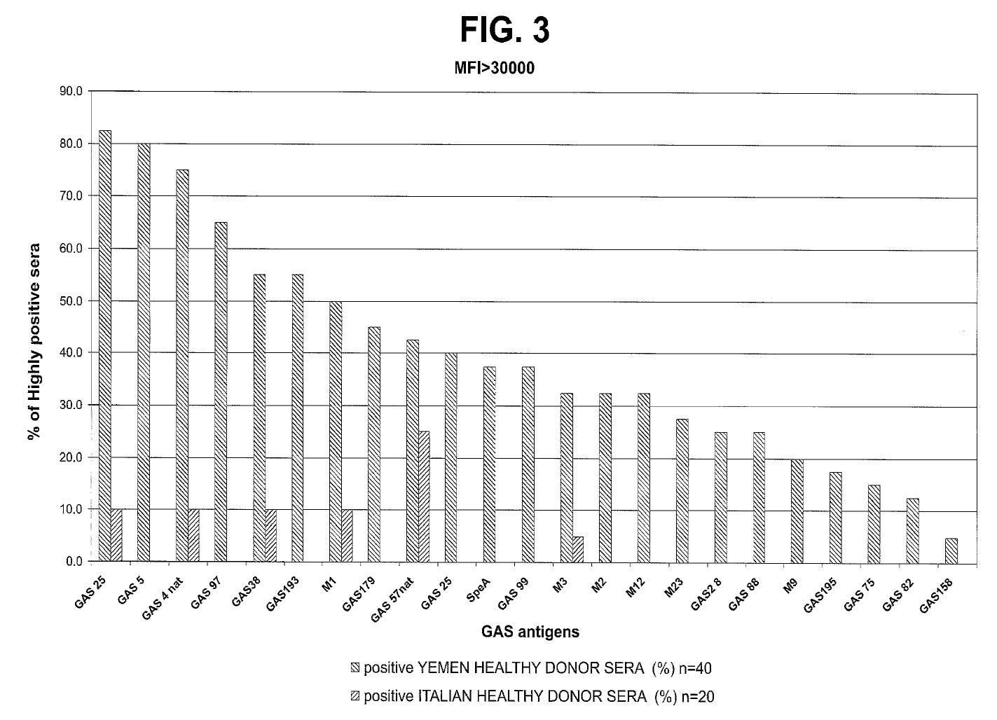

Anti GAS antibody responses in populations belonging to the two different geographical areas were investigated using 40 sera from Yemen healthy blood donors and 20 healthy donors from Italy. FIG. 3 reports the percentage of healthy donor sera with high responses to GAS antigens. As shown, background antistreptococcal responses are much higher in Yemenite than in Italian samples, both in terms of number of highly recognized antigens and as number of highly positive sera.

### Differential Immuno Reactivity of GAS Antigens Between Healthy and RHD Yemenite Patients

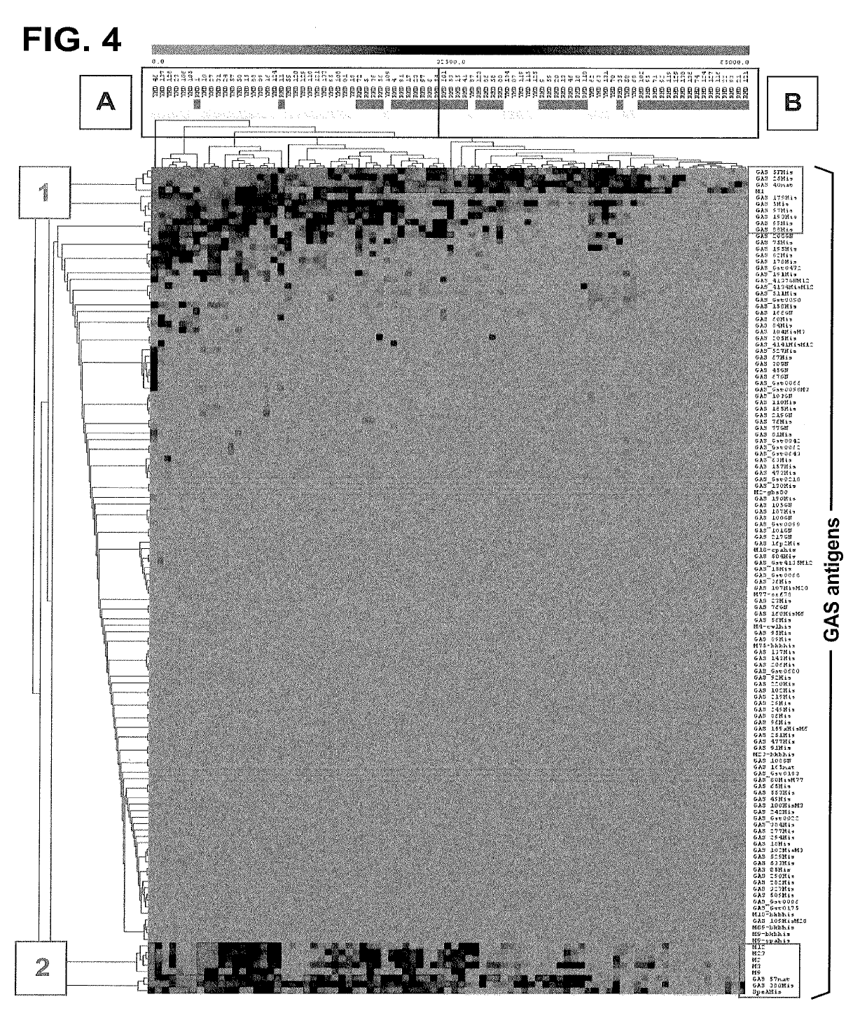

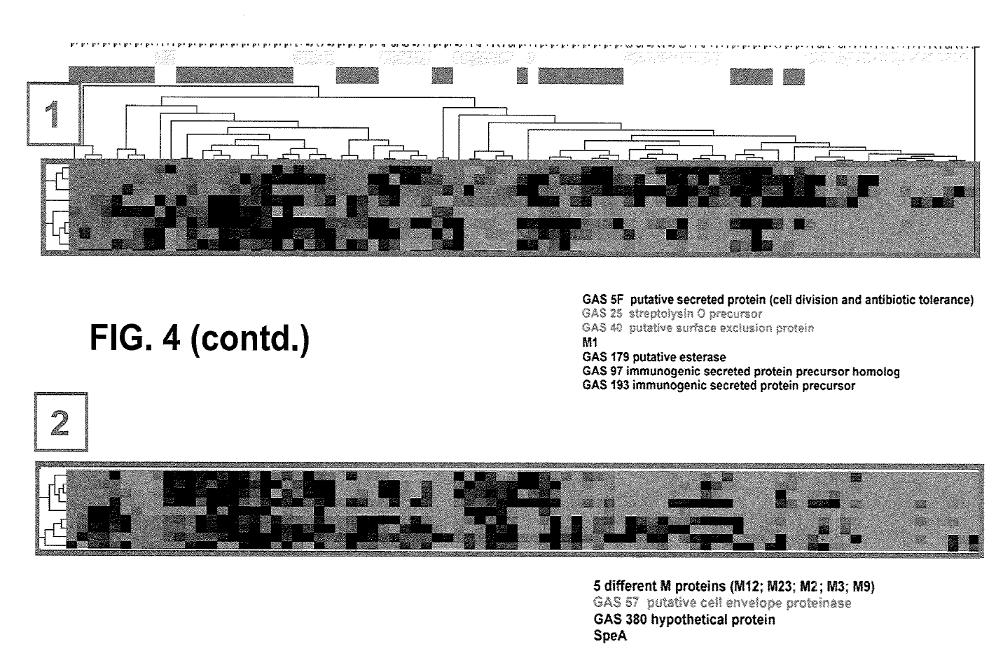

We compared the immuno reactivity of the 40 YHD (dark grey in FIG. 4) and 43 RHD (light grey in FIG. 4) age matched selected human sera. To define the antigen recognition patterns of the two groups of sera, normalized FI (MFI) values were subjected to unsupervised bi-dimensional hierarchical clustering using dedicated software (TIGR Multiexperiment Viewer (MeV) software (http://www.tigr.org/software/tm4/mev.html).

The clustered view of the antibody recognition profiles identified two major groups of highly recognized antigens (1 and 2 in FIG. 4). Group 1 included GAS5F (putative secreted protein), GAS25 (streptolysin O precursor), GAS40 (putative surface exclusion protein), M1, GAS179 (putative esterase), GAS97 (immunogenic secreted protein precursor homolog), GAS193 (uimmunogenic secreted protein precursor). Group 2 included 5 different M proteins (M12, M23, M2, M3 and M9), GAS57 (putative cell envelope proteinase), GAS380 (hypothetical protein) and SpeI.

Furthermore, as shown in FIG. 4, this clustering analysis distributed the sera from higher reactivity (on the left) to lower reactivity (on the right). Indeed, two main groups of sera could be distinguished, a highly reactive group which comprised mainly sera from healthy donors (A in left of FIG. 4) and second group displaying lower reactivity and including mainly sera from RHD patients (group B in FIG. 4).

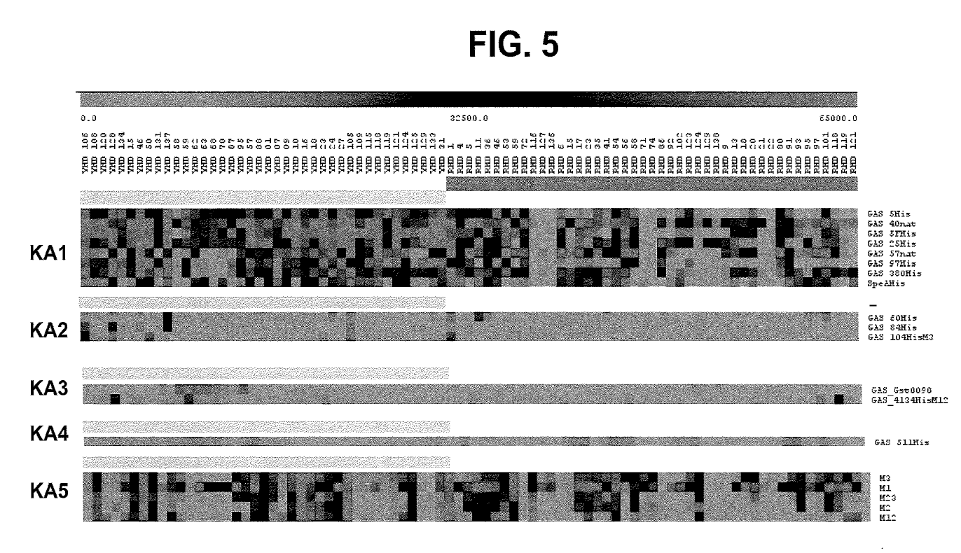

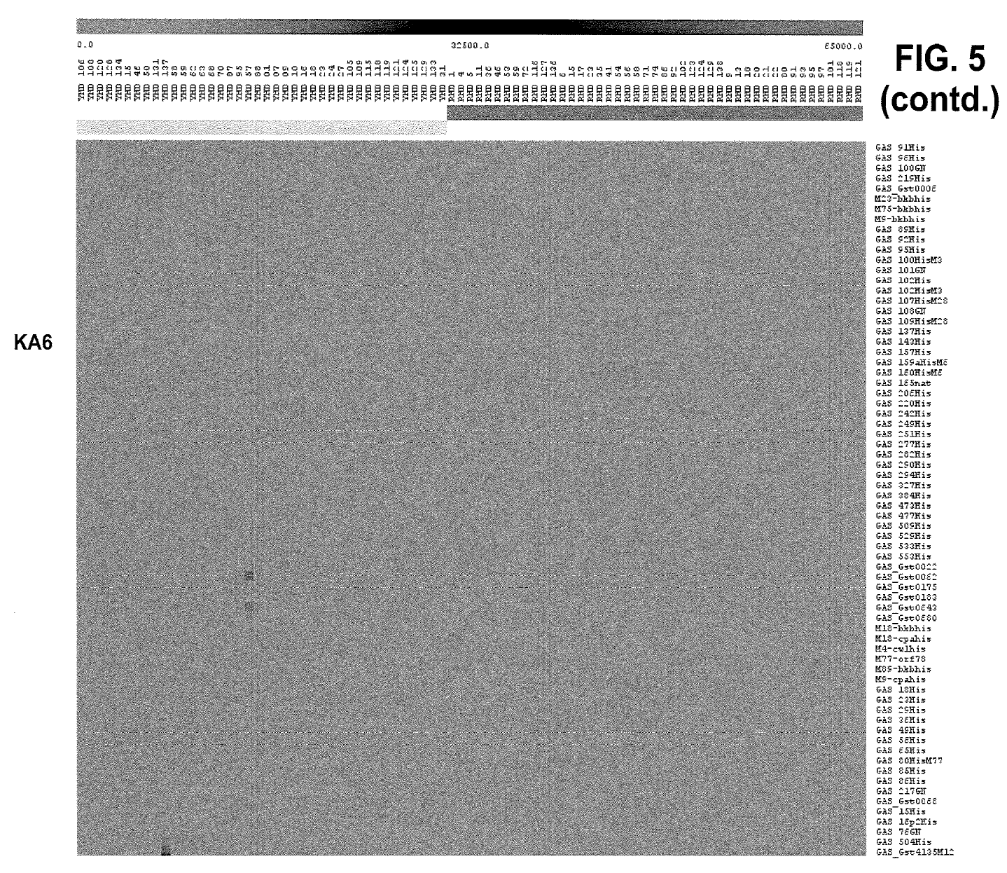

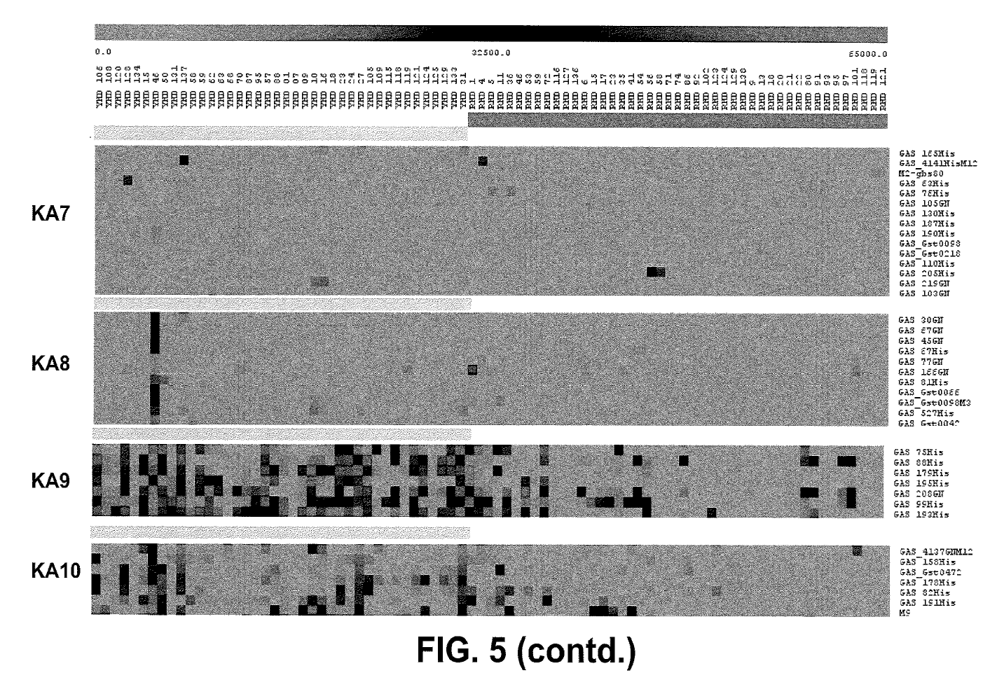

We then applied the K-mean cluster analysis in order to classify the GAS antigens present in the chip in 10 clusters (KA1 to KA10 in FIG. 5) eliciting similar recognition patterns. k-mean clustering is a statistical method aimed at partitioning n observations into k clusters in which each observation belongs to the cluster with the nearest mean. It is similar to the expectation-maximization algorithm for mixtures of Gaussians in that they both attempt to find the centers of natural clusters in the data.

As shown in FIG. 5, four of the antigen clusters (numbers KA 1, KA 5, KA 9, KA 10) identified contained antigens with higher fluorescence values than the remaining clusters. Interestingly, even though the clustering was mono-dimensional, i.e. was intended for antigen classification and not for classification of the sera, we observed in that in cluster KA 1, the most reactive sera comprised a large number of YHD. This observation suggested the presence in the chip of a group of antigens, the reactivity of which allows discrimination between sera derived from healthy donors and sera from RHD patients.

We then tried to define in a more precise manner the group of antigens allowing us to discriminate between healthy and cardiopathic patients. For this purpose, sera were further classified on the basis of their recognition profiles to different groups of antigens using a mono-dimensional hierarchical clustering analysis.

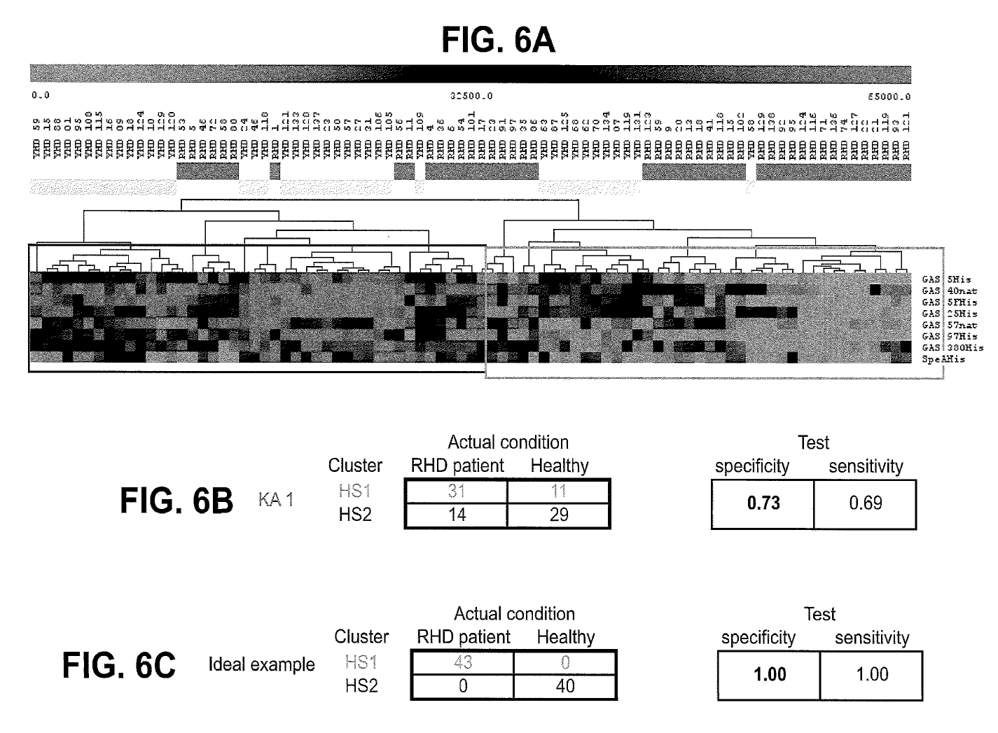

This type of analysis was first applied to the group of antigens included in cluster KA1 of FIG. 5, allowing the definition of two sera clusters at the first hierarchical level, HS 1 and HS2, corresponding to violet (HS1) and blue (HS2) boxes in FIG. 6A. The numbers of healthy sera and patient sera present or absent in each of the two clusters is reported in FIG. 6B. As shown, most of the YHD can be found on the high reactivity, blue, HS2 cluster, while the majority of RHD sera are found in the low reactivity, violet HS1 cluster. The ability to distinguish between the two sera populations using this type of test was defined in terms of specificity and sensitivity. FIG. 6C shows the ideal theoretical example of maximum specificity and sensitivity (values of 1). As shown, for this particular group of antigens we obtained specificity and sensitivity values of 0.73 and 0.69.

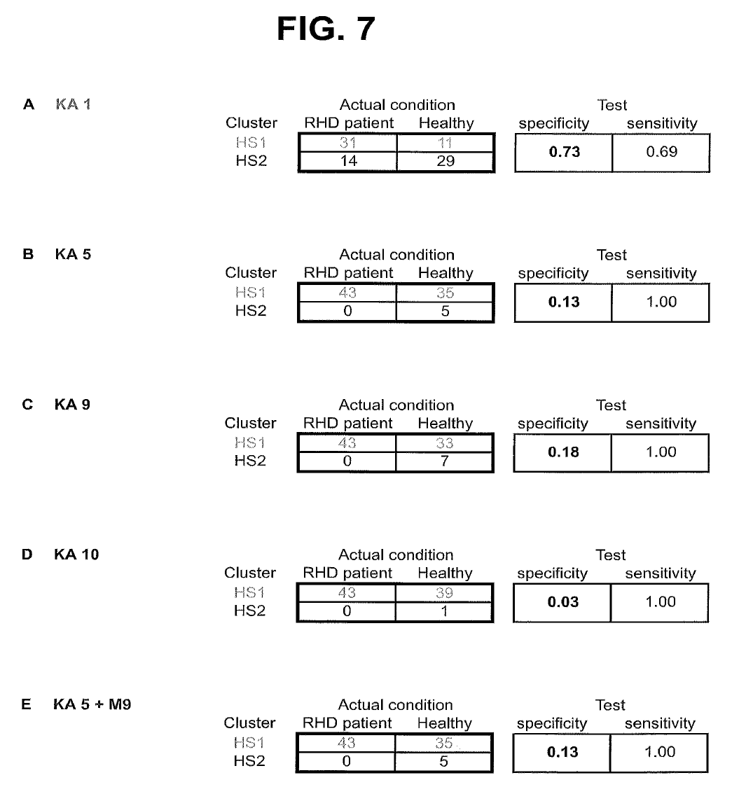

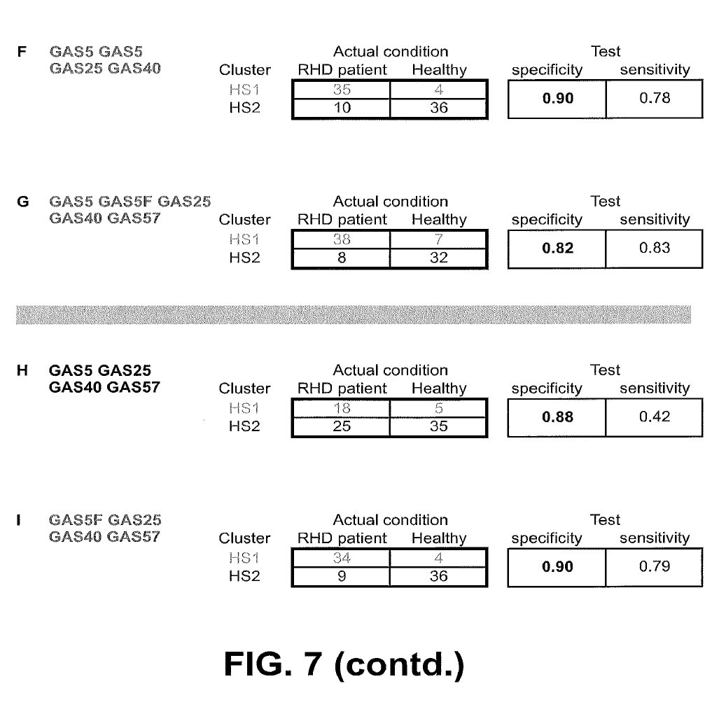

The same type of analysis was applied to the antigens in clusters KA5, KA9, KA10, and KA5+M9. The same type of analysis was also applied to other groups of antigens, including GAS5+GAS5F+GAS25+GAS40, GAS5+GAS5F+GAS25+GAS40+GAS57, GAS5+GAS25+GAS40+GAS57, GAS5F+GAS25+GAS40+GAS57. The results are summarized in FIG. 7 A-I.

As shown, the cluster yielding highest specificity and sensitivity values comprised antigens in cluster KA1 (GAS5, GAS40, GAS5F, GAS57, GAS97, GAS380 and SpeA), and antigens GAS5, GAS25, GAS40 and GAS57, while lowest values were obtained for the cluster including GAS M variants.

### Discussion

We believe that this type of analysis using antigens GAS5, GAS25, GAS40 and GAS57 could set the basis for a more precise diagnosis of RHD and allow prediction of whether a patient with ARF is likely to develop RHD, thus guiding medical professionals to decide on the best prophylactic therapy for ARF patients.

### REFERENCES

[1] Winter et al., (1991) Nature 349:293-99
[2] U.S. Pat. No. 4,816,567.
[3] Inbar et al., (1972) Proc. Natl. Acad. Sci. U.S.A. 69:2659-62.
[4] Ehrlich et al., (1980) Biochem 19:4091-96.
[5] Huston et al., (1988) Proc. Natl. Acad. Sci. U.S.A. 85:5897-83.
[6] Pack et al., (1992) Biochem 31, 1579-84.
[7] Cumber et al., (1992) J. Immunology 149B, 120-26.
[8] Riechmann et al., (1988) Nature 332, 323-27.
[9] Verhoeyan et al., (1988) Science 239, 1534-36.
[10] GB 2,276,169.
[11] U.S. Pat. No. 6,355,271.
[12] WO00/23105.
[13] Vaccine Design . . . (1995) eds. Powell & Newman. ISBN: 030644867X. Plenum.
[14] WO90/14837.
[15] WO90/14837.
[16] Podda & Del Giudice (2003) Expert Rev Vaccines 2:197-203.
[17] Podda (2001) Vaccine 19: 2673-2680.
[18] Vaccine Design: The Subunit and Adjuvant Approach (eds. Powell & Newman) Plenum Press 1995 (ISBN 0-306-44867-X).
[19] Vaccine Adjuvants: Preparation Methods and Research Protocols (Volume 42 of Methods in Molecular Medicine series). ISBN: 1-59259-083-7. Ed. O'Hagan.
[20] WO2008/043774.
[21] Allison & Byars (1992) Res Immunol 143:519-25.
[22] Hariharan et al. (1995) Cancer Res 55:3486-9.
[23] US-2007/014805.
[24] US-2007/0191314.
[25] Suli et al. (2004) Vaccine 22(25-26):3464-9.
[26] WO95/11700.
[27] U.S. Pat. No. 6,080,725.
[28] WO2005/097181.
[29] WO2006/113373.
[30] Han et al. (2005) Impact of Vitamin E on Immune Function and Infectious Diseases in the Aged at Nutrition, Immune functions and Health EuroConference, Paris, 9-10 Jun. 2005.
[31] U.S. Pat. No. 6,630,161.
[32] U.S. Pat. No. 5,057,540.
[33] WO96/33739.
[34] EP-A-0,109,942.
[35] WO96/11711.
[36] WO00/07621.
[37] Barr et al. (1998) Advanced Drug Delivery Reviews 32:247-271.
[38] Sjolanderet et al. (1998) Advanced Drug Delivery Reviews 32:321-338.
[39] Niikura et al. (2002) Virology 293:273-280.
[40] Lenz et al. (2001) J Immunol 166:5346-5355.
[41] Pinto et al. (2003) J Infect Dis 188:327-338.
[42] Gerber et al. (2001) J Virol 75:4752-4760.
[43] WO03/024480.
[44] WO03/024481.
[45] Gluck et al. (2002) Vaccine 20:B10-B16.
[46] EP-A-0689454.
[47] Johnson et al. (1999) Bioorg Med Chem Lett 9:2273-2278.
[48] Evans et al. (2003) Expert Rev Vaccines 2:219-229.
[49] Meraldi et al. (2003) Vaccine 21:2485-2491.
[50] Pajak et al. (2003) Vaccine 21:836-842.
[51] Kandimalla et al. (2003) Nucleic Acids Research 31:2393-2400.
[52] WO02/26757.
[53] WO99/62923.
[54] Krieg (2003) Nature Medicine 9:831-835.
[55] McCluskie et al. (2002) FEMS Immunology and Medical Microbiology 32:179-185.
[56] WO98/40100.
[57] U.S. Pat. No. 6,207,646.
[58] U.S. Pat. No. 6,239,116.
[59] U.S. Pat. No. 6,429,199.
[60] Kandimalla et al. (2003) Biochemical Society Transactions 31 (part 3):654-658.
[61] Blackwell et al. (2003) J Immunol 170:4061-4068.
[62] Krieg (2002) Trends Immunol 23:64-65.
[63] WO01/95935.
[64] Kandimalla et al. (2003) BBRC 306:948-953.
[65] Bhagat et al. (2003) BBRC 300:853-861.
[66] WO03/035836.
[67] WO01/22972.
[68] Schellack et al. (2006) Vaccine 24:5461-72.
[69] Kamath et al. (2008) Eur J Immunol 38:1247-56.
[70] Riedl et al. (2008) Vaccine 26:3461-8.
[71] WO95/17211.
[72] WO98/42375.
[73] Beignon et al. (2002) Infect Immun 70:3012-3019.
[74] Pizza et al. (2001) Vaccine 19:2534-2541.
[75] Pizza et al. (2000) Int J Med Microbiol 290:455-461.
[76] Scharton-Kersten et al. (2000) Infect Immun 68:5306-5313.
[77] Ryan et al. (1999) Infect Immun 67:6270-6280.
[78] Partidos et al. (1999) Immunol Lett 67:209-216.
[79] Peppoloni et al. (2003) Expert Rev Vaccines 2:285-293.
[80] Pine et al. (2002) J Control Release 85:263-270.
[81] Tebbey et al. (2000) Vaccine 18:2723-34.
[82] Domenighini et al. (1995) Mol Microbiol 15:1165-1167.
[83] WO99/40936.
[84] WO99/44636.
[85] Singh et all (2001) J Cont Release 70:267-276.
[86] WO99/27960.
[87] U.S. Pat. No. 6,090,406.
[88] U.S. Pat. No. 5,916,588.
[89] EP-A-0626169.
[90] WO99/52549.
[91] WO01/21207.
[92] WO01/21152.
[93] Andrianov et al. (1998) Biomaterials 19:109-115.
[94] Payne et al. (1998) Adv Drug Delivery Review 31:185-196.
[95] U.S. Pat. No. 4,680,338.
[96] U.S. Pat. No. 4,988,815.
[97] WO92/15582.
[98] Stanley (2002) Clin Exp Dermatol 27:571-577.
[99] Wu et al. (2004) Antiviral Res. 64(2):79-83.
[100] Vasilakos et al. (2000) Cell Immunol. 204(1):64-74.
[101] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[102] Jones (2003) Curr Opin Investig Drugs 4:214-218.
[103] WO03/011223.
[104] Johnson et al. (1999) Bioorg Med Chem Lett 9:2273-2278.
[105] Evans et al. (2003) Expert Rev Vaccines 2:219-229.
[106] WO2004/060308.
[107] WO2004/064759.
[108] U.S. Pat. No. 6,924,271.
[109] US2005/0070556.
[110] U.S. Pat. No. 5,658,731.
[111] U.S. Pat. No. 5,011,828.
[112] WO2004/87153.
[113] U.S. Pat. No. 6,605,617.
[114] WO02/18383.
[115] WO2004/018455.
[116] WO03/082272.
[117] Wong et al. (2003) J Clin Pharmacol 43(7):735-42.
[118] US2005/0215517.
[119] Dyakonova et al. (2004) Int Immunopharmacol 4(13):1615-23.
[120] FR-2859633.
[121] Signorelli & Hadden (2003) Int Immunopharmacol 3(8):1177-86.
[122] WO2004/064715.
[123] De Libero et al, Nature Reviews Immunology, 2005, 5: 485-496
[124] U.S. Pat. No. 5,936,076.
[125] Oki et al, J. Clin. Investig., 113: 1631-1640
[126] US2005/0192248
[127] Yang et al, Angew. Chem. Int. Ed., 2004, 43: 3818-3822
[128] WO2005/102049
[129] Goff et al, J. Am. Chem., Soc., 2004, 126: 13602-13603
[130] WO03/105769
[131] Cooper (1995) Pharm Biotechnol 6:559-80.
[132] WO99/11241.
[133] WO94/00153.
[134] WO98/57659.
[135] European patent applications 0835318, 0735898 and 0761231.
[136] WO2006/110603.

1-13. (canceled)

14. A method of treating or preventing rheumatic heart disease (RHD) associated with S. pyogenes (GAS) infection, comprising administering to a patient in need thereof at least one GAS antigen comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

15. A method of diagnosing rheumatic heart disease (RHD) associated with S. pyogenes (GAS) infection in a patient, or of identifying a patient at risk of developing RHD associated with GAS infection, comprising:
(a) contacting a biological sample from a patient with at least one GAS antigen under conditions appropriate for binding of any antibodies present in the biological sample to the at least one GAS antigen, and
(b) comparing the reactivity of antibodies in the biological sample from the patient to the at least one GAS antigen with the reactivity of antibodies in a control biological sample from a healthy individual to the at least one GAS antigen,
wherein a lower reactivity in the biological sample from the patient compared to the control biological sample from a healthy individual is indicative that the patient is suffering from RHD associated with GAS infection or that the patient is at risk of developing RHD associated with GAS infection.

16. The method of claim 15 further comprising administering the at least one GAS antigen to the patient.

17. The method of claim 15, wherein the at least one GAS antigen is selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

18. The method of claim 17, wherein the sample is contacted with 1, 2, 3, 4, 5, 6, 7, or 8 of the GAS antigens.

19. The method of claim 17, wherein the sample is contacted with three GAS antigens selected from the group consisting of:
three GAS antigens comprising the amino acid sequences SEQ ID NO:1, SEQ ID NO:2, and SEQ ID NO:3, respectively;
three GAS antigens comprising the amino acid sequences SEQ ID NO:1, SEQ ID NO:3, and SEQ ID NO:4, respectively;
three GAS antigens comprising the amino acid sequences SEQ ID NO:1, SEQ ID NO:4, and SEQ ID NO:5, respectively;
three GAS antigens comprising the amino acid sequences SEQ ID NO:2, SEQ ID NO:3, and SEQ ID NO:4, respectively;
three GAS antigens comprising the amino acid sequences SEQ ID NO:2, SEQ ID NO:4, and SEQ ID NO:5, respectively; and
three GAS antigens comprising the amino acid sequences SEQ ID NO:3, SEQ ID NO:4, and SEQ ID NO:5, respectively.

20. The method of claim 17, wherein the sample is contacted with four GAS antigens selected from the group consisting of:
four GAS antigens comprising the amino acid sequences SEQ ID NO:1, SEQ ID NO:2, SEQ ID NO:3, and SEQ ID NO:4, respectively;
four GAS antigens comprising the amino acid sequences SEQ ID NO:2, SEQ ID NO:3, SEQ ID NO:4, and SEQ ID NO:5, respectively; and
four GAS antigens comprising the amino acid sequences SEQ ID NO:1, SEQ ID NO:3, SEQ ID NO:4, and SEQ ID NO:5, respectively.

21. The method of claim 17 wherein the sample is contacted with five GAS antigens comprising the amino acid sequences of SEQ ID NO:1, SEQ ID NO:2, SEQ ID NO:3, SEQ ID NO:4, and SEQ ID NO:5.

22. The method of claim 15, wherein the biological sample is a serum sample.

23. The method of claim 15, wherein the biological sample is from an adolescent or from a child.

24. The method of claim 15, wherein the GAS antigens are displayed on one or more protein arrays.

25. A protein array comprising at least two GAS antigens, each comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

26. A kit comprising:
a protein array comprising at least two S. pyogenes (GAS) antigens, each comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8; and
instructions for the use of the array in the diagnosis of patients having or at risk of developing rheumatic heart disease associated with GAS infection.


SEQUENCE LISTING (10 sequences)

SEQ ID NO: 1 - protein, 398 aa, Streptococcus pyogenes
MKKRILSAVLVSGVTLGAATTVGAEDLSTKIAKQDSIISNLTTEQKAAQNQVSALQAQVSSLQSEQDKLTARNTELEALSKRFEQEIKALTSQIVARNEKLKNQARSAYKNNETSGYINALLNSKSISDVVNRLVAINRAVSANAKLLEQQKADKVSLEEKQAANQTAINTIAANMAMAEENQNTLRTQQANLVAATANLALQLASATEDKANLVAQKEAAEKAAAEALAQEQAAKVKAQEQAAQQAASVEAAKSAITPAPQATPAAQSSNAIEPAALTAPAAPSAGPQTSYDSSNTYPVGQCTWGAKSLAPWAGNNWGNGGQWAYSAQAAGYRTGSTPMVGAIAVWNDGGYGHVAVVVEVQSASSIRVMESNYSGRQYIADHRGWFNPTGVTFIYPH

SEQ ID NO: 2 - protein, 175 aa, Streptococcus pyogenes
AAAEALAQEQAAKVKAQEQAAQQAASVEAAKSAITPAPQATPAAQSSNAIEPAALTAPAAPSAGPQTSYDSSNTYPVGQCTWGAKSLAPWAGNNWGNGGQWAYSAQAAGYRTGSTPMVGAIAVWNDGGYGHVAVVVEVQSASSIRVMESNYSGRQYIADHRGWFNPTGVTFIYPH

SEQ ID NO: 3 - protein, 571 aa, Streptococcus pyogenes
MSNKKTFKKYSRVAGLLTAALIIGNLVTANAESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 4 - protein, 873 aa, Streptococcus pyogenes
MDLEQTKPNQVKQKIALTSTIALLSASVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQRALKAGVVMLAAVGLTGFRFRKESK

SEQ ID NO: 5 - protein, 1647 aa, Streptococcus pyogenes
MEKKQRFSLRKYKSGTFSVLIGSVFLVMTTTVAADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKALPSTGEKMGLKLRIVGLVLLGLTCVFSRKKSTKD

SEQ ID NO: 6 - protein, 503 aa, Streptococcus pyogenes
MNKNKLLRVAMLLSLLAPTAESMTVLAQDVMLETHKATTNETSDSSSKEENNKNAAPTTSDKTDQGPLDASAETNSNSLVNADDKKRSDSSQSAIGSSDNKAEAENQVDDKSTDHSKSTDHSKPTDQPKPSPSKVDTAPASSLSKQLPEARTPIQSLSPYVSDLDLSEIDIPSVNTYAAYVEHWSGKNAYTHHLLSRRYGIKADQIDSYLKSTGIAYDSTRINGEKLLQWEKKSGLDVRAIVAIAMSESSLGTQGIATLLGANMFGYAAFDLDPTQASKFNDDSAIVKMTQDTIIKNKNSNFALQDLKAAKFSRGQLNFASDGGVYFTDTTGSGKRRAQIMEDLDKWIDDHGGTPAIPAELKVQSSASFASVPAGYKLSKSYDVLGYQASSYAWGQCTWYVYNRAKELGYQFDPFMGNGGDWKYKVGYALSKTPKVGYAISFAPGQAGADGTYGHVSIVEDVRKDGSILISESNCIGLGKISYRTFTAQQAEQLTYVIGKSKN

SEQ ID NO: 7 - protein, 995 aa, Streptococcus pyogenes
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGSLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 8 - protein, 221 aa, Streptococcus pyogenes
QQDPDPSQLHRSSLVKNLQNIYFLYEGDPVTHENVKSVDQLLSHDLIYNVSGPNYDKLKTELKNQEMATLFKDKNVDIYGVEYYHLCYLCENAERSACIYGGVTNHEGNHLEIPKKIVVKVSIDGIQSLSFDIETNKKMVTAQELDYKVRKYLTDNKQLYTNGPSKYETGYIKFIPKNKESFWFDFFPEPEFTQSKYLMIYKDNETLDSNTSQIEVYLTTK

SEQ ID NO: 9 - protein, 865 aa, Streptococcus pyogenes
ADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKN

SEQ ID NO: 10 - protein, 298 aa, Streptococcus pyogenes
SVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQ